(19) United States
(12) Patent Application Publication
(10) Pub. No.: US 2022/0071944 A1
(43) Pub. Date: Mar. 10, 2022

(54) Combination Treatment for Solid Tumors Using Docetaxel and a CYP3A Inhibitor

(71) Applicant: Modra Pharmaceuticals B.V., HN Amsterdam (NL)

(72) Inventors: Jacob Hendrik Beijnen, HN Amsterdam (NL); Johannes Henricus Schellens, HN Amsterdam (NL)

(21) Appl. No.: 17/416,946

(22) PCT Filed: Dec. 18, 2019
(86) PCT No.: PCT/EP2019/086125

§ 371 Date: Jun. 21, 2021

(30) Foreign Application Priority Data
Dec. 21, 2018 (EP) 18215488.0

(51) Int. Cl. A61K 31/337; A61K 31/427

(52) U.S. Cl. CPC A61K 31/337; A61K 31/427

(57) ABSTRACT

Treatments of cancers involve a wide range of treatment. The current invention relates to chemotherapy of tumors using taxanes, in particular docetaxel. More in particular it relates to achieving efficacious doses of orally administered doses docetaxel whilst maintaining acceptable safety. By providing novel means and methods, combining oral docetaxel with a CYP3A inhibitor, the inventors have established improved treatments of cancer, said methods and means providing for an improved safety profile of docetaxel as compared with the standard of treatment for docetaxel, while at the same time allowing to obtain efficacious levels of docetaxel to eradicate cancer cells.

### BACKGROUND OF THE INVENTION

### The Field of the Invention

The invention relates to chemotherapy of tumors using taxanes, in particular docetaxel. More in particular it relates to achieving efficacious doses of orally administered doses docetaxel whilst maintaining acceptable toxicity.

### Background

Treatments of cancers involve a wide range of treatment. Treatments include i.a. surgery, radiation therapy, chemotherapy, immunotherapy and cell therapy. Often, cancer treatments include a combination of different modes of treatments, comprising combinations of different therapeutic agents. As part of first line chemotherapy, docetaxel, a taxane, is used widely in the treatment of various cancers. Docetaxel is a cytotoxic agent, its main mode of action is understood to involve interference with microtubule assembly and disassembly, resulting in inhibiting mitotic cell division. The recommended dosage is a three-weekly intravenous administration, with a dose in the range of 75-100 mg/m2 of body surface area. Docetaxel is used in the treatment of a variety of cancers, which include breast, lung, prostate, gastric, head and neck, and ovarian cancer. While having the potential to benefit patients, improving life expectancy and quality of life, the use of docetaxel comes along with significant side effects. Typical side effects include i.a. neutropenia, a high risk of infections, thrombocytenia, anemia, alopecia, fluid retention, diarrhoea, nail toxicity, peripheral sensory neurotoxicity and infusion related reactions. Hence, the recommended mode of use involves a restricted number of cycles, usually 4-6 cycles, of docetaxel. In addition, standard premedication with high dose dexamethasone is needed every cycle.

### SUMMARY OF THE INVENTION

Docetaxel administered intravenously as a chemotherapeutic agent is approved and in use for the treatment of a variety solid tumors. Variability has been observed in patients with regard to response to treatment. The current inventors now sought to provide for improved means and methods for utilizing docetaxel in the treatment of cancer. In particular, the inventors provide for a different route of administration of docetaxel, i.e., orally, combined with a cytochrome P450 isoenzyme CYP3A (CYP3A) inhibitor, in order to achieve docetaxel exposure levels that are comparable or at least comparable to a standard of care treatment for docetaxel. By providing methods and uses combining oral docetaxel with a CYP3A inhibitor, the inventors have established improved means and methods for the treatment of cancer, said methods and means providing for an improved safety profile of docetaxel as compared with the standard of care treatment for docetaxel, while at the same time allowing to exert control to obtain efficacious anti-tumor levels of docetaxel exposure. Also, the methods and means of the invention allow the avoidance of use of standard premedication with high dose dexamethasone, which is recommended in every cycle during the standard of care treatment for docetaxel. Hence, provided herein is a combination therapy in the treatment of cancer wherein said docetaxel is to be administered orally in combination with a CYP3A inhibitor, whereby the dose of the CYP3A inhibitor is sufficient to obtain docetaxel exposure levels in the tumor tissue which are comparable, or at least comparable, to a standard of care treatment for docetaxel. In one embodiment a use is provided for a combination therapy of docetaxel to be administered orally in combination with a CYP3A inhibitor, in the treatment of cancer, whereby the dose of docetaxel is adjusted to compensate for increased clearance of docetaxel in subjects having cancer. The dosages of docetaxel and CYP3A can be selected for the cancer to be treated to obtain sufficient docetaxel exposure levels of the tumor tissue that are comparable, or at least comparable to a standard of care treatment for docetaxel. Alternatively, CYP3A activity of subjects and/or docetaxel plasma levels of subjects that are to undergo the combination treatment and/or are in combination treatment, in accordance with the invention, can be determined to adjust docetaxel and/or CYP3A inhibitor dosage to control and monitor plasma levels of docetaxel that are sufficient to maintain exposure levels in the tumor tissue at least comparable to a standard of care treatment for docetaxel.

### BRIEF DESCRIPTION OF THE DRAWINGS

In FIG. 1 a plot is shown presenting AUC (average AUCs in h*ng/mL by dose level) of ritonavir (RTV) to ModraDoc006 (docetaxel). It shows that exposure of Modrodoc006 appears to be highly correlated to overall Ritonavir AUC (and dose).

In FIG. 2A a plot is shown indicating similar or moderately higher levels of docetaxel AUC are obtained in patients as compared with IV. In FIG. 2B a plot is shown with the AUC of ritonavir.

In FIG. 3 a plot is shown of docetaxel AUC and number of cycles. Length of treatment appears to be trending longer in patients in a target docetaxel range.

FIGS. 4, 5A, 5B and 6 represent updates of FIGS. 1, 2A, 2B, and 3, respectively.

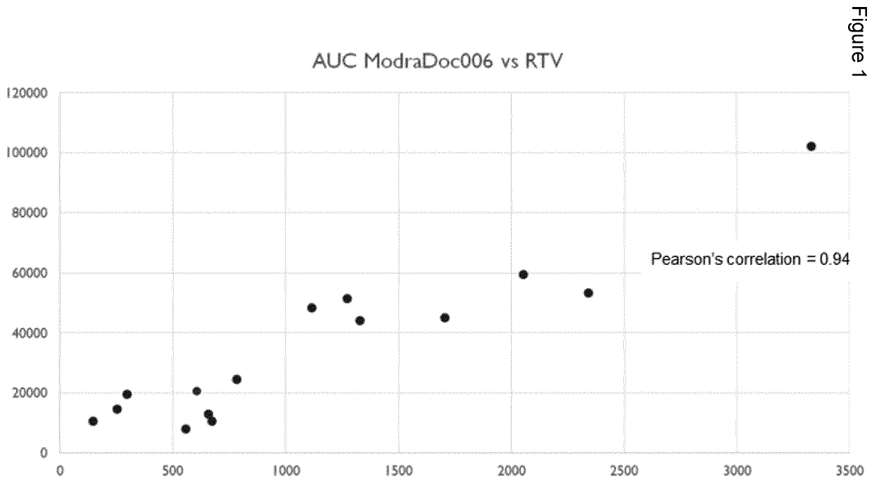

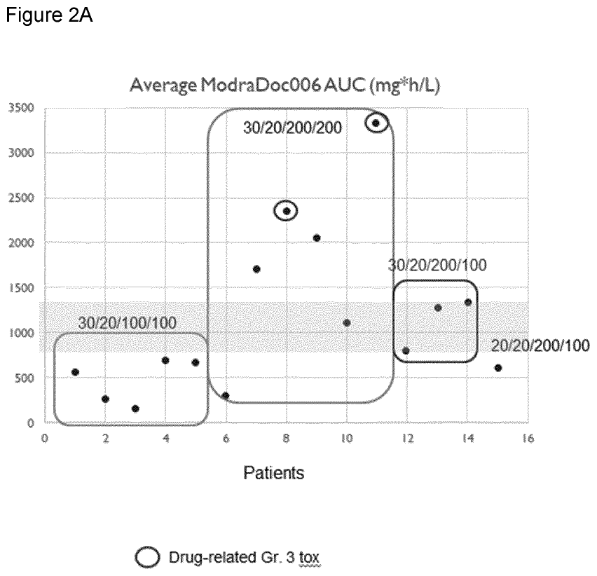

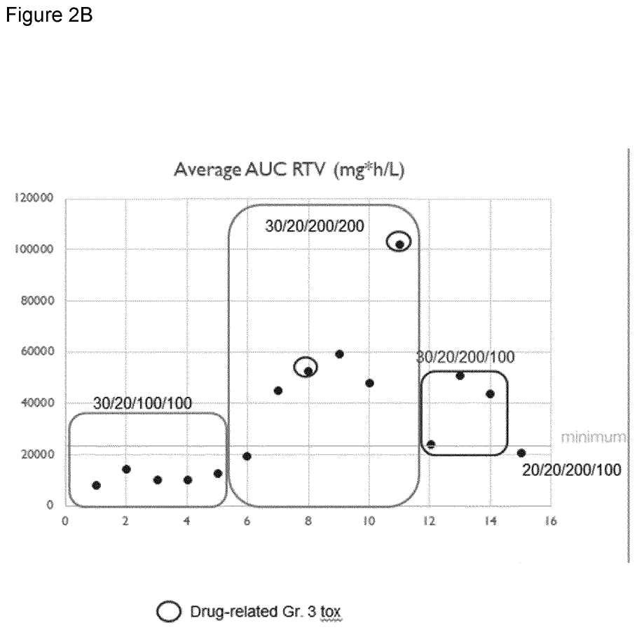

In FIG. 4 a plot is shown presenting AUC (average AUCs in h*ng/mL by dose level) of ritonavir (RTV) to ModraDoc006 (docetaxel). It shows that exposure of Modrodoc006 appears to be highly correlated to overall Ritonavir AUC (and dose).

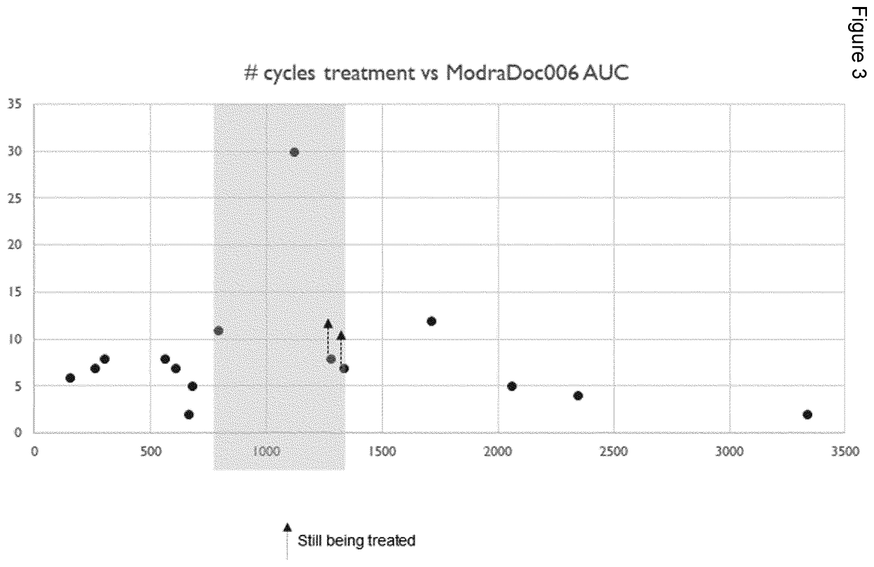

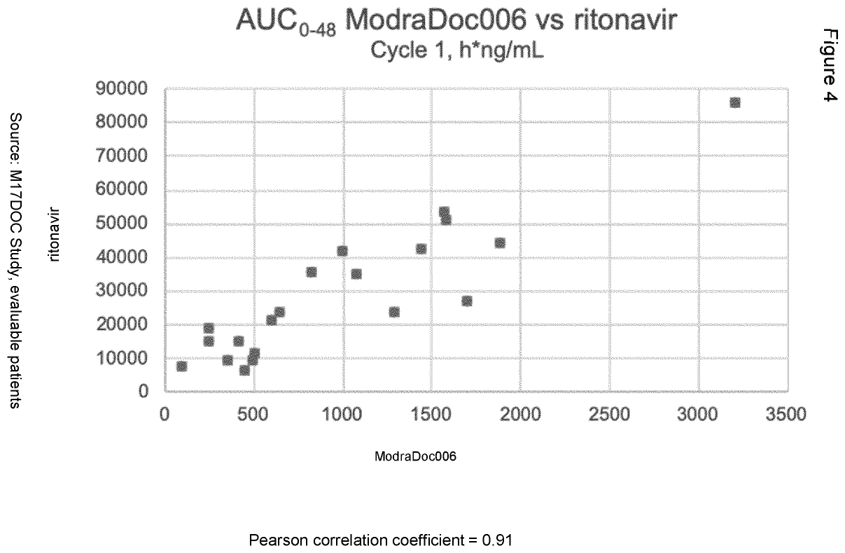

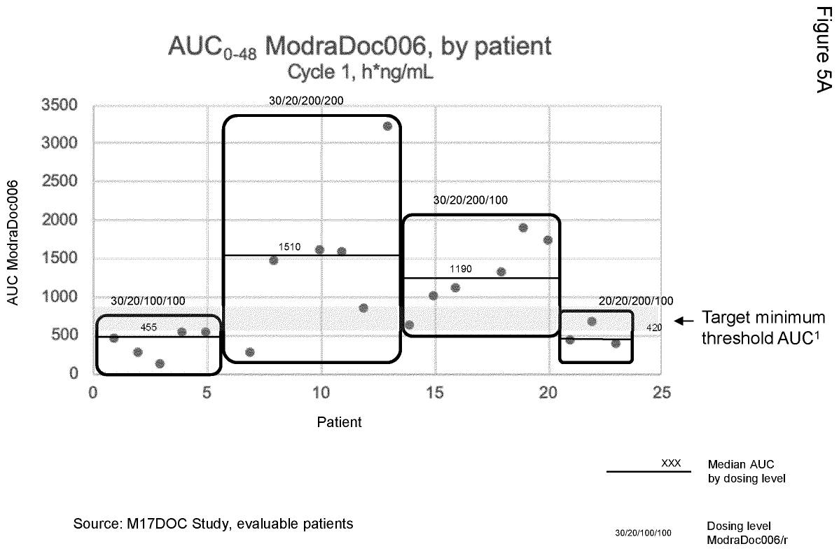

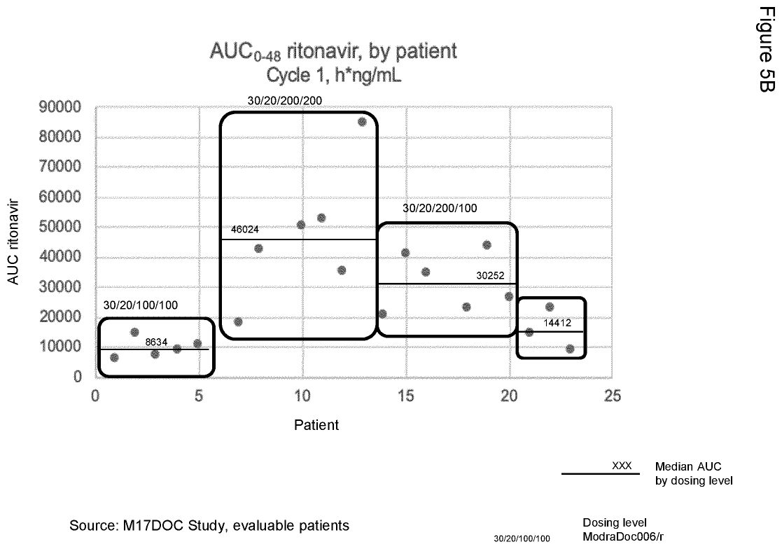

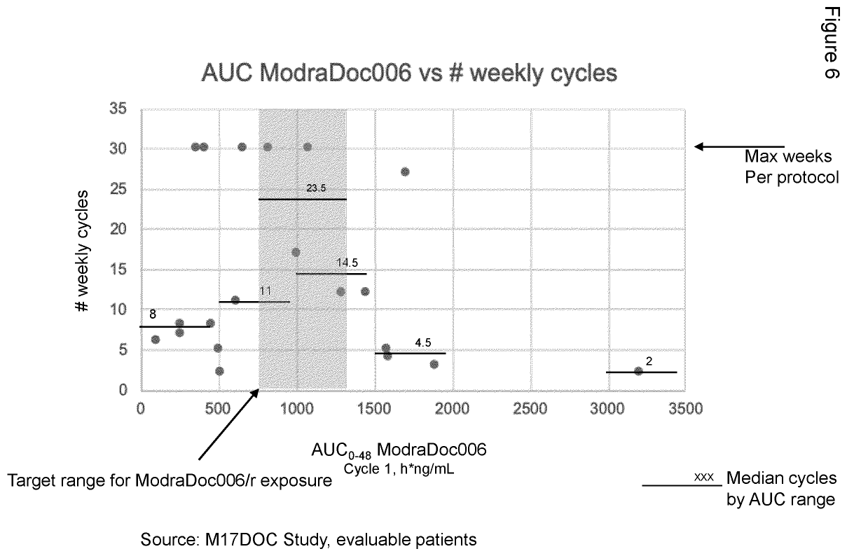

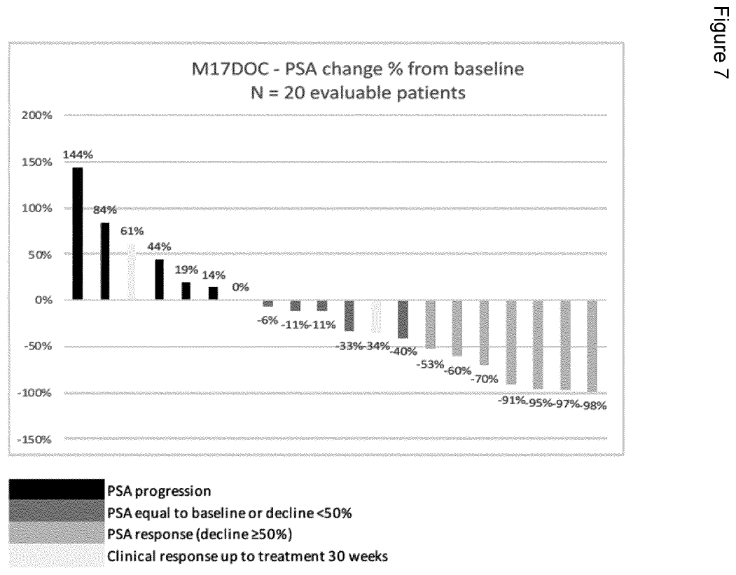

In FIG. 5A a plot is shown indicating similar or moderately higher levels of docetaxel AUC are obtained in patients with ModraDoc006/r as compared with IV.

The target minimum AUC threshold in mCRPC patients is highlighted and ranges from about 600-800 h*ng/mL. This represents, at its lower limit, the weekly AUC of IV docetaxel in mCRPC patients (1820/3=±600 h*ng/mL [dividing q3w AUC of 1820 by 3 to yield weekly equivalent]. Source: De Vries Schultink et al, “Neutropenia and docetaxel exposure in metastatic castration-resistant prostate cancer patients: A meta-analysis and evaluation of a clinical cohort”, Cancer Medicine, February 2019. At its upper limit, this represents 1418*55%=±800 h*ng/ML—where 1418 represents the AUC of ModraDoc006/r in its phase I study N10BOM. 55% (1820/3300) represents the ratio of AUCs for IV docetaxel in mCPRC patients vs other tumors (De Vries Schultink et al, “Neutropenia and docetaxel exposure in metastatic castration-resistant prostate cancer patients: A meta-analysis and evaluation of a clinical cohort”, Cancer Medicine, February 2019).

In FIG. 5B a plot is shown with the AUC of ritonavir.

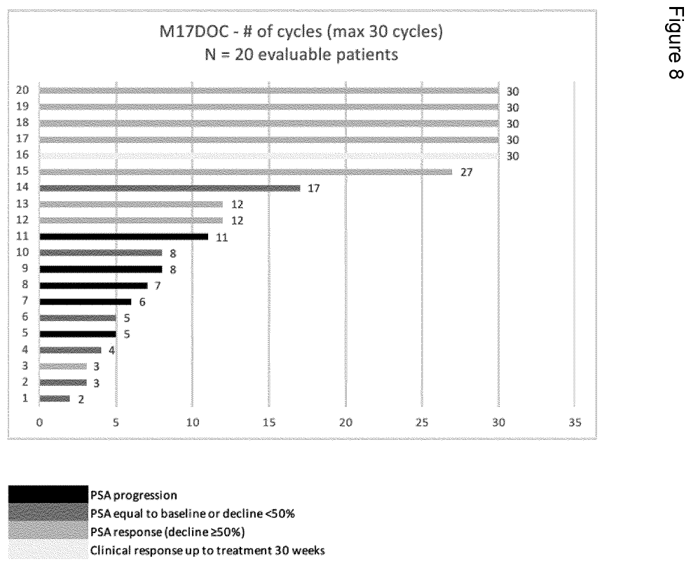

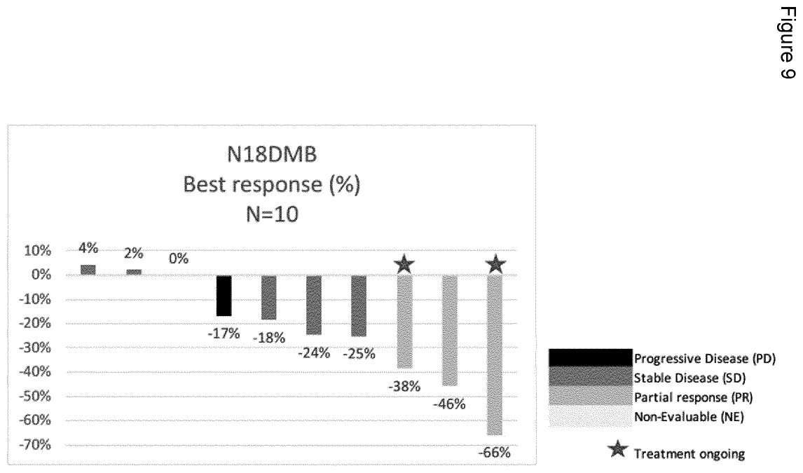

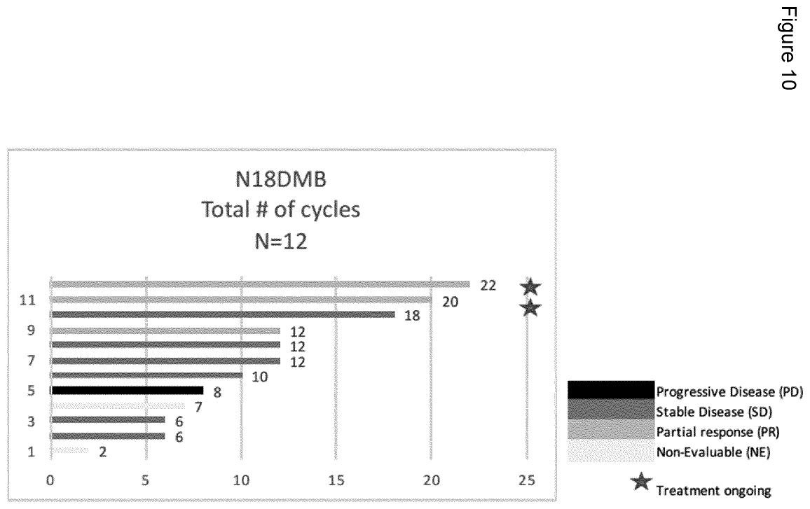

In FIG. 6 a plot is shown of docetaxel AUC and number of cycles. Length of treatment appears to be trending longer in patients in a target docetaxel range between 500-1500 h*ng/mL.

In FIG. 7 a plot is shown of PSA (prostate specific antigen) change % from baseline in a multicenter clinical phase IB study in mCRPC (M17DOC) of evaluable patients. Patients were scored as PSA progression (black bars); PSA equal to baseline or decline (<50%) (dark grey bars); PSA response (decline ≥50%) (medium grey bars); clinical response (pain reduction) up to the maximum treatment period permitted in the protocol of 30 weeks (light grey bars).

In FIG. 8 a plot is shown of the number of treatment cycles (with a maximum of 30) in a multicenter clinical phase IB study in mCRPC (M17DOC) of evaluable patients. Patients were scored, as PSA progression (black bars); PSA equal to baseline or decline (<50%) (dark grey bars); PSA response (decline ≥50%) (medium grey bars); clinical response (pain reduction) up to the maximum treatment period permitted in the protocol of 30 weeks (light grey bars).

In FIG. 9 a plot is shown of best responders in a multicenter phase IIA study in HER2-metastatic breast cancer (mBC) (N18DMB) of 10 patients evaluable for response with regard to tumor measurements, a negative % indicating the percentage decrease of tumor size. Patients were scored as having progression disease (PD) (black bars); Stable disease (SD) (dark grey bars); partial response (PR) (medium grey bars); or non evaluable (NE) (light grey bars). Patients indicated with a star have treatment ongoing.

In FIG. 10 a plot is shown of the total number of cycles in in a multicenter phase IIA study in HER2-metastatic breast cancer (mBC) (N18DMB) of 12 patients evaluable for safety evaluation. Patients scores were also indicated as having progression disease (PD) (black bars); Stable disease (SD) (dark grey bars); partial response (PR) (medium grey bars); or non evaluable (NE) (light grey bars). Patients indicated with a star have treatment ongoing.

### DETAILED DESCRIPTION

Docetaxel administered intravenously as a chemotherapeutic agent is approved and in use for the treatment of a variety solid tumors. With regard to response to treatment variability in patients has been observed. The current inventors now sought to provide for improved means and methods for utilizing docetaxel in the treatment of cancer. In particular, the inventors provide for a different route of administration of docetaxel, i.e., orally, in order to achieve docetaxel exposure levels that are comparable to a standard of care treatment for docetaxel. When docetaxel is administered intravenously, high peak levels of docetaxel can be measured in blood plasma of subjects (which can also be measured in serum, or whole blood). The inventors now have established that the high peak levels are associated with the toxicity in the standard of care treatments. When docetaxel is administered orally and combined with a CYP3A inhibitor, such high peak levels of docetaxel can be largely avoided. Importantly, the current inventors established that with orally administered docetaxel combined with a cytochrome P450 3A4 (and P450 3A5) (CYP3A) inhibitor, docetaxel exposure levels can be obtained that are comparable, or at least comparable, to a standard of care treatment for docetaxel, this results in efficacious doses of docetaxel for the treatment of a cancer while at the same time maintaining an acceptable toxicity. This is of importance for combination therapies, in which combinations of anticancer treatments are combined.

Side effects that may be controlled or reduced in the current treatment include neutropenia. Such neutropenia may be a febrile neutropenia. Neutropenia is an abnormally low concentration of neutrophils in the blood. Neutropenia is usually diagnosed by determining the absolute neutrophil count in the blood. As a reference, a healthy range of neutrophil count in the blood can be defined as having 1500-4000 cells per microliter of blood. Neutropenia may be diagnosed when the level of neutrophils is below 1500 cells per microliter of blood. Assays to determine neutrophil counts are widely available as part of e.g., a complete blood count analysis as part of routine laboratory testing. Accordingly, in the current invention, the incidence of neutropenia is significantly reduced in the patient population while concomitantly providing for an effective treatment of the cancer in patients. Hence, preferably, in the method of treatment of a cancer in a patient, the side effect neutropenia is controlled or reduced. Other side effects that may be controlled or reduced are thrombocytopenia, neuropathy, alopecia, fluid retention, neurotoxicity, and/or nail toxicity.

Further side effects that may be avoided by using oral administration of docetaxel in accordance with the invention include infusion-related reactions due to e.g., excipients (i.a. Tween-80, ethanol) used in intravenous formulations of docetaxel. Corticosteroids, such as dexamethasone, are used as a prophylaxis for such infusion-related reactions in current intravenous docetaxel treatments. By using orally administered docetaxel, which does not require corticosteroid prophylaxis, toxicity that may be associated with (long-term) treatment with corticosteroids may be avoided as well.

As used herein, the oral administration of docetaxel to a subject includes any route through the mouth of introducing or delivering to a subject the agent to perform its intended function. Suitable pharmaceutical compositions for oral administration includes liquids, tablets or capsules. Capsules and tablets may have an enteric coating, such that docetaxel is released from the capsules or tablets in the intestine. Capsules and tablets may be formulated in an extended release formulation such that docetaxel is released over an extended period, e.g., several hours or more, e.g., during the time spend in the intestinal tract. Tablets and capsules may thus be formulated such that the agent is released therefrom gradually. Tablets and capsules may be formulated such that the agent is released in the stomach or intestine. Tablets and capsules may be formulated such that the agent is released in the stomach and intestine. Administration includes self-administration and the administration by another.

Pharmaceutical compositions of this invention may comprise docetaxel, or pharmaceutically acceptable salts and esters thereof, and/or a CYP3A inhibitor, such as ritonavir, (or pharmaceutically acceptable salts and esters thereof) together with any pharmaceutically acceptable carrier, adjuvant or vehicle. Suitable preparations and/or pharmaceutical compositions for oral administrations include formulations as described in WO2009027644, WO2010020799 and Moes et al. Drug Deliv. Transl. Res. 2013) which are incorporated herein in its entirety by reference. Any suitable preparation for oral administration can be contemplated.

The current invention may not be restricted to oral administration of docetaxel. Any administration of docetaxel via the gastrointestinal tract may be contemplated. Hence, enteral administration can be contemplated herein instead of oral administration. Preferably, enteral administration is in the form of capsules, tablets, and suppositories. Docetaxel administration via a suppository may be advantageous, as bioavailability may be improved as compared with oral administration. This is because with oral administration, after passing the stomach and intestine, docetaxel is delivered to the liver via the portal vein. By enteral administration, the barriers that metabolize docetaxel in the first-pass may be avoided. Any enteral administration may suffice, as long as peak levels are avoided and effective plasma levels are obtained, as defined herein.

For many anticancer drugs such as docetaxel, cytochrome P450 represents a main oxidative drug metabolizing enzyme system. Cytochrome P450 (CYP) iso-enzymes, in particular CYP3A4, which also may include CYP3A5, (referred to as CYP3A herein) are highly expressed in the liver and intestine. Intestinal extraction and metabolism of docetaxel by this enzyme system plays an important role in limiting oral bioavailability. As part of the metabolic route transporters also play a role. By the transport of compounds, such as docetaxel, in and out of the cell, the compound is provided as a substrate to the CYP3A4 and/or CYP3A5 enzymes. For example, the P-glycoprotein (P-gp, MDR1, ABCB1) plays a role in the metabolic route and transport of docetaxel. Hence, any compound that may have an effect on the metabolic route of docetaxel to thereby inhibit metabolizing docetaxel may be considered a suitable CYP3A inhibitor. Such compounds have an effect on CYP3A4 and/or CYP3A5, and on P-glycoprotein (Er-jiaWang et al, Chem. Res. Toxicol. 2001; Wacher et al., Mol Carc. 1995), or may have distinct action on either CYP3A4 and/or CYP3A5, and on P-glycoprotein (Er-jiaWang et al, Chem. Res. Toxicol. 2001). Suitable CYP3A inhibitors may thus have an effect on both CYP3A4 (and CYP3A5) and P-glycoprotein. Suitable CYP3A inhibitors may thus have an effect on CYP3A4 and/or CYP3A5. Suitable CYP3A inhibitors may have an effect on P-glycoprotein. Hence, a CYP3A inhibitor is defined herein as a compound capable of reducing CYP3A4 and CYP3A5 metabolism in the cell. Said compound preferably is a pharmaceutical compound. Preferably, a CYP3A inhibitor is selected that inhibits CYP3A4, such as e.g., ritonavir. Ritonavir inhibits CYP3A5 and P-glycoprotein as well. Selective inhibition of CYP3A4 is highly preferred.

In the methods of treatments of a cancer in a patient comprising orally administered docetaxel as described herein, preferably the plasma levels of docetaxel are at least partially controlled by administering a CYP3A inhibitor. The use of a CYP3A inhibitor accordingly assisting in transporting docetaxel from the stomach and/or intestine to the bloodstream, by reducing and/or inhibiting CYP3A4 and/or CYP3A5 activity in the cell. The use of a CYP3A inhibitor can thus provide for increased bioavailability of docetaxel. Such bioavailability may be increased, while not substantially increasing the peak levels of docetaxel. Hence, the use of a CYP3A inhibitor allows for the use of a lower dosage of oral docetaxel as effective plasma levels of docetaxel can be increased as compared with not using a CYP3A inhibitor. Alternatively, the use of a CYP3A inhibitor allows for the use of less frequent dosing of oral docetaxel, as effective plasma levels with the area under the curve as defined herein can be more efficiently obtained as compared with not using a CYP3A inhibitor.

Hence, in a method in accordance with the invention, the plasma levels of docetaxel are at least partially controlled by administering a CYP3A inhibitor. As said, the oral administration of docetaxel is to be combined with the use of a CYP3A inhibitor. Any CYP3A inhibitor may suffice, e.g., a suitable CYP3A inhibitor may be potent CYP3A inhibitors selected from the group consisting of boceprevir, claritromycine, erytromycine, indinavir, itraconazole, ketoconazole, posaconazole, ritonavir, saquinavir en voriconazole. Preferably a CYP3A inhibitor is used that has the least side effects. Most preferably, the CYP3A inhibitor that is combined with oral administration of docetaxel is ritonavir. Preferably, the CYP3A inhibitor for use in a combination therapy in accordance with the invention, comprises ritonavir administered in a dosage of 100 mg or 200 mg, or an equivalent dosage of another suitable CYP3A inhibitor. One can easily establish the suitable dosage for any other suitable inhibitor, as one can compare the effect of the CYP3A inhibitor ritonavir in a subject and select another CYP3A inhibitor and establish the dosage thereof that obtains the same effect. The effect being defined as the effect on docetaxel plasma levels (AUC) and/or peak plasma levels as obtained with the dosage of ritonavir used.

It is understood that in the methods and uses in accordance of the inventions, any additional use of compounds, including foods and further pharmaceuticals, that may have an impact on CYP3A activity, are preferably avoided as such foods may have an effect on the levels of docetaxel achieved in the plasma of subjects being treated. Hence, whichever potent CYP3A inhibitor is selected for the combined treatment with docetaxel, the further use of inhibitors of CYP3A by the subjects receiving treatment needs to be avoided as this may result in too high peak levels of docetaxel and/or to high area under the curves. Examples of further inhibitors that are preferably avoided are e.g., HIV Antivirals: indinavir, nelfinavir and saquinavir; Anti-microbial agents: clarithromycin, itraconazole, ketoconazole, nefazodone, telithromycin, erythromycin, fluconazole, chloramphenicol, ciprofloxacin, norfloxacin and voriconazole; Cardiac agents: verapamil, diltiazem, cimetidine and miodarone; other agents such as fluvoxamine; and also foods, such as star fruit and grapefruit juice. Conversely, preferably in the methods and uses of the invention, the use of compounds, including foods and further pharmaceuticals, that may induce CYP3A activity in the subjects receiving treatment, is preferably avoided as well, as such use may result in too high peak levels of docetaxel in plasma. Inducers of CYP3A that are preferably avoided are: HIV Antivirals: efavirenz and nevirapine; Other agents such as: barbiturates, carbamazepine, modafinil, nevirapine, oxcarbazepine, phenobarbital, phenytoin, pioglitazone, rifabutin, rifampicin and also St. John's wort.

In one embodiment, in the method in accordance with the invention said CYP3A inhibitor is simultaneously administered with docetaxel. It is understood that simultaneous administration can comprise separate administrations, e.g., in separate pharmaceutical preparations. For example, one pharmaceutical preparation suitable for oral administration comprising docetaxel and another pharmaceutical preparation comprising the CYP3A inhibitor, such as ritonavir. The pharmaceutical preparation comprising ritonavir preferably also being orally administered. It is understood that simultaneous administration can comprise one pharmaceutical preparation comprising both docetaxel and the CYP3A inhibitor, such as ritonavir. Docetaxel and the CYP3A inhibitor, can also be administered separately from each other. When they are administered separately, the CYP3A inhibitor is preferably administered before docetaxel, and, more preferably, within approximately 60 minutes before docetaxel is administered. Simultaneously, as used herein, means administration of the docetaxel or CYP3A inhibitor within e.g., approximately 20 minutes, more preferably within 15 minutes, more preferably within 10 minutes, even more preferably within 5 minutes, most preferably within 2 minutes of the CYP3A inhibitor or docetaxel. Generally, the CYP3A inhibitor is preferably orally administered simultaneously with administering oral docetaxel as this provides for optimal compliance in self-administration by subjects receiving treatment.

While CYP3A activity, in e.g., the liver and in the intestine, can have an effect on the exposure levels that are obtained in the blood after oral administration of docetaxel, which can be controlled by the use of CYP3A inhibitors and/or selecting a suitable docetaxel dosage, there may also be other unknown causes that have an effect on the exposure levels of docetaxel that can be obtained after oral administration of docetaxel. As shown in the examples herein, docetaxel clearance was apparently increased in mCRPC as compared with other solid tumors. Hence, increasing the docetaxel dose in patients having increased clearance of docetaxel to obtain higher exposure levels of docetaxel could be beneficial to such patients. However, increasing the docetaxel dose through the use of intravenous administrations of docetaxel will provide for an unacceptable high peak concentration in blood plasma, making this unacceptable with a standard of care treatment. In contrast, by using orally administered docetaxel combined with a CYP3A inhibitor in accordance with the invention, exposure levels of docetaxel can be well controlled avoiding unacceptable high peak concentrations. Hence, the inventors have established that the dose of docetaxel and/or the dose of the CYP3A inhibitor used can be selected such that when using orally administered docetaxel combined with a CYP3A inhibitor, the exposure levels obtained with docetaxel can be rendered highly efficacious for the treatment of a cancer and provide for an acceptable toxicity to subjects. Thus, the present invention provides docetaxel for use in a combination therapy in the treatment of cancer wherein said docetaxel is to be administered orally in combination with a CYP3A inhibitor, whereby the dose of the CYP3A inhibitor is sufficient to obtain docetaxel exposure levels of the tumor tissue which are comparable to a standard of care treatment for docetaxel. Docetaxel for use in a combination therapy in the treatment of cancer wherein said docetaxel is to be administered orally in combination with a CYP3A inhibitor, whereby the dose of the CYP3A inhibitor is sufficient to obtain docetaxel exposure levels in the tumor tissue which are [at least] comparable to a standard of care treatment for docetaxel.

In another embodiment, docetaxel is provided for use in a combination therapy in the treatment of cancer, wherein said docetaxel is to be administered orally in combination with a CYP3A inhibitor, whereby the dose of docetaxel is adjusted to compensate for increased clearance of docetaxel in subjects having cancer.

In another embodiment, a CYP3A inhibitor is provided for use in a combination therapy in the treatment of cancer wherein said CYP3A inhibitor is to be administered in combination with an oral formulation of docetaxel, whereby the dose of the CYP3A inhibitor is sufficient to obtain docetaxel exposure levels of the tumor tissue which are comparable, or at least comparable to a standard of care treatment for docetaxel. In yet another embodiment, a CYP3A inhibitor is provided for use in a combination therapy in the treatment cancer, wherein said CYP3A inhibitor is to be administered in combination with an oral formulation of docetaxel, whereby the dose of the CYP3A inhibitor is sufficient to substantially diminish the increased clearance of docetaxel in subjects having cancer.

Hence, the current inventors have established that when using a combination of a CYP3A inhibitor and orally administered docetaxel, sufficient docetaxel exposure levels can be obtained that can eradicate cancer cells while at the same time having acceptable toxicity. Sufficient docetaxel exposure levels can be obtained e.g., by having a sufficient dose of CYP3A and/or an adjusted dose of orally administered docetaxel. As long as the exposure levels that are obtained are comparable or are at least comparable to a standard of care treatment for docetaxel, such a combination of orally administered docetaxel and CYP3A inhibitor is contemplated herein.

The standard of care treatment for docetaxel as used herein is defined as an intravenous administration of a recommended dose of docetaxel. The recommended dose of docetaxel is usually between 75 mg/m2 and 100 mg/m2 every 3 weeks, (milligrams of docetaxel per square meter of body surface area of a subject). A recommended dose for a non-small-cell lung cancer, a breast cancer, a gastric cancer, a head and neck cancer or a prostate cancer is usually 75 mg/m2 every 3 weeks. The recommended dose may also be 35 mg/m2 per week. Docetaxel exposure levels of the tumor tissue can be defined herein as the area under the curve as obtained when administering docetaxel intravenously and corresponding with an effective standard of care treatment for docetaxel. It is understood that this may not define the actual docetaxel level of the tissue, as docetaxel is measured in plasma.

The area under the curve (AUC; ng*h/mL) is determined in the first 48 hours after the administration of docetaxel, during which the docetaxel concentration in blood plasma can be measured at several timepoints, and the surface of the area under the curve can be calculated from the plotted values. Plasma levels of docetaxel can be measured by methods known in the art (Hendrikx et al. J. Chrom. B, 2011), which may include liquid chromatography and mass spectrometry methods, such as e.g., described in the examples. Plasma is a blood component, it is understood that instead of measuring docetaxel in blood plasma, one can also determine levels of docetaxel in whole blood or in serum. Measurements of docetaxel, e.g., peak levels and area under the plasma concentration-time curve, in short area under the curve (AUC) herein are defined relative to (blood) plasma but can easily be recalculated to corresponding peak levels in whole blood or serum. In general, preferably, the AUC is within the range of 500-2500 ng·h/mL. Preferably, the AUC is at least 500 ng·h/mL, at least 600 ng·h/mL, at least 800 ng·h/mL, more preferably at least 1000 or 1200. Preferably, the AUC is at most 2500 ng·h/mL. at most 2250 ng·h/mL, at most 2000 ng·h/mL, at most 1800 ng·h/mL, at most 1700 ng·h/mL, more preferably at most 1500 ng·h/mL. More preferably, the AUC may be within the range of 800-1400 ng·h/mL. Herein, plasma concentration-time curve, area under the curve, or AUC, with reference to docetaxel are used interchangeably and refer to the area under the curve in the first 48 hours (ng·h/mL) after the administration of docetaxel.

Practically these dosages can be reached by orally administering 50 mg of docetaxel in two doses in one day (e.g., 30 mg morning and 20 mg evening) once a week.

Preferably, provided is docetaxel or the CYP3A inhibitor for use in accordance with the invention as described herein, wherein the cancer is a solid tumor. Preferably, said use of docetaxel or the CYP3A inhibitor is a use wherein the solid tumor is a non-small-cell lung cancer, a gastric cancer, a breast cancer, a head and neck cancer or a prostate cancer. Said solid tumors being preferred because docetaxel has been shown to be highly efficacious in these cancers, with the oral administration route of docetaxel combined with CYP3A providing for improved and/or acceptable toxicity to subjects having these cancers.

Most preferably, said cancer is a prostate cancer. The treatment of prostate cancer can involve the use of hormonal therapy, e.g., using androgen deprivation therapy. The prostate cancer may not respond to hormonal therapy, such prostate cancer termed hormonal refractory prostate cancer (HRPC). The prostate cancer may respond to hormonal therapy, such a prostate cancer termed hormonal sensitive prostate cancer (HSPC). Such patients may also have metastases, or may develop metastases during treatment. Such cancers referred to as mHRPC or mHSPC (m indicating metastatic). A prostate cancer treatment may include castration. In any case, prostate cancer generally involves the reduction of testosterone in the body to very low levels. As shown in the examples, in patients that have mCRPC, clearance of docetaxel as determined in plasma is apparently increased relative to patients having solid tumors that are not mCRPC. Such patients have very low levels of testosterone. Patients that receive intravenously administered docetaxel concurrent with androgen deprivation therapy in an early phase of the disease experience more toxicity relative to prostate cancer patients that receive intravenously administered docetaxel in a later phase of the disease. Hence, in prostate cancer treatments that involves the use of hormonal therapy, which involves androgen inhibitors, reduced docetaxel plasma levels by increased clearance of docetaxel can be anticipated as well, but such an increased clearance may need first to be established after hormonal therapy has commenced. The dosage of docetaxel and/or CYP3A inhibitor may therefore be adjusted in the early phase of the treatment of prostate cancer to compensate for relatively lower plasma levels in such patients as compared with patients with mCRPC. Conversely, plasma levels of docetaxel may need first to be established, e.g., by administering a first oral dosage of docetaxel combined with a CYP3A inhibitor in accordance with the invention and determining plasma concentrations (such as AUC) of docetaxel, to confirm that the same dosages as suitable for mCRPC can be administered. In any case, the dosages suitable for mCRPC may also be suitable for HSPC, HRPC, mHRPC, mHSPC or CRPC (i.e., non-metastatic). In a preferred embodiment, dosages suitable for mCRPC are also selected for the treatment of mHSPC.

In a preferred embodiment, the prostate cancer is a metastatic castration-resistant prostate cancer (mCRPC). Hence, in a further embodiment, in accordance with the invention, a method is provided for the treatment of a metastatic castration-resistant prostate cancer (mCRPC), comprising orally administering an effective dose of docetaxel, in combination with a CYP3A inhibitor, whereby the dose of the CYP3A inhibitor is sufficient to obtain docetaxel exposure levels of the tumor tissue which is [at least] comparable to a standard of care treatment of docetaxel given intravenously every three weeks, absent any increased clearance by enzymatic activity, in particular CYP3A. Preferably, said standard care treatment referred to is a cancer which is not mCRPC. In another embodiment, a method in accordance with the invention is provided for the treatment of a metastatic castration-resistant prostate cancer (mCRPC), comprising orally administering an effective dose of docetaxel, in combination with a CYP3A inhibitor, whereby the dose of docetaxel is adjusted to compensate for the increased clearance of docetaxel in subjects having mCRPC. In yet another further embodiment, a method is provided for the treatment of a metastatic castration-resistant prostate cancer (mCRPC), comprising orally administering an effective dose of docetaxel, in combination with a CYP3A inhibitor, whereby the dose of the CYP3A inhibitor is sufficient to substantially diminish the increased clearance of docetaxel in subjects having mCRPC.

In one embodiment, docetaxel or a CYP3A inhibitor for use in a combination therapy in accordance with the invention is provided, docetaxel is administered orally in a weekly dosage, wherein the docetaxel exposure levels are comparable to a standard of care treatment resulting in area under the curve of 600-1800 ng·h/mL, more preferably of 1000-1500 ng·h/mL. The oral administration of docetaxel in this embodiment is at a dose that is similar to the recommended dose, but e.g., given in a more frequent interval, i.e., every week instead of e.g., three-weekly. Because of the use of an oral administration combined with a CYP3A inhibitor, this route of administration results in at least comparable exposure to docetaxel in the subjects receiving treatment. Because of the route of administration however, the peak levels of docetaxel are highly reduced, thereby allowing for more frequent dosing while maintaining at least comparable exposure to docetaxel.

Hence, as shown in the examples, the docetaxel exposure levels obtained (as determined with AUC as described herein) in accordance with the invention can be comparable to a standard of care treatment, or may be selected to be higher as compared with the standard of care treatment (see i.a., FIG. 5A). Hence, higher levels may be advantageous, and in the uses and methods in accordance with the invention, achieving at least comparable levels of docetaxel may be preferred.

Docetaxel preferably is administered on a bi-daily basis once a week. The weekly dose is split so that a subject takes, for example, on one day a first dose in the morning and the second dose in the evening once a week. This has the effect of decreasing peak levels of docetaxel in plasma which may aid reducing side effects, while allowing to obtain a sufficient area under the curve. It also may increase the time of systemic exposure of the drug. In a preferred embodiment, the methods, or uses, in accordance with the invention comprise docetaxel being administered bidaily weekly, meaning that on one day every week, docetaxel is administered twice, e.g., within an 8-16 hours interval. As long as the dosing interval and/or dosage of docetaxel and the CYP3A inhibitor, such as ritonavir, is selected that allows to provide for docetaxel exposure levels in the tumor tissue which are comparable or at least comparable to a standard of care treatment for docetaxel, such dosing interval and/or dosage may be contemplated.

In another embodiment, for the treatment of solid tumors, docetaxel is administered orally at a weekly dosage of 50 mg, as such a dosage as shown in the example section can provide for docetaxel exposure levels in tumor tissues that are comparable or at least comparable to a standard of care treatment for docetaxel. Such an administration is preferably at a bi-daily weekly schedule. Hence, a bi-daily weekly schedule for the treatment of solid tumors is provided herein for the treatment of solid tumors, wherein docetaxel is administered on one day a week a first administration at a dosage of 30 mg docetaxel with 100 mg ritonavir and a second administration at a dosage of 20 mg docetaxel with 100 mg ritonavir.

In another embodiment, for the treatment of solid tumors, docetaxel is administered orally at a weekly dosage of 40 mg, as such a dosage as shown in the example section can provide for docetaxel exposure levels in tumor tissues that are comparable or at least comparable to a standard of care treatment for docetaxel. Such an administration is preferably at a bi-daily weekly schedule. Hence, a bi-daily weekly schedule for the treatment of solid tumors is provided herein for the treatment of solid tumors, wherein docetaxel is administered on one day a week a first administration at a dosage of 20 mg docetaxel with 200 mg ritonavir and a second administration at a dosage of 20 mg docetaxel with 100 mg ritonavir.

In one embodiment, a bidaily weekly schedule is provided for the treatment of cancer, wherein docetaxel is administered on one day a week a first administration at a dosage of 30 mg docetaxel with 200 mg ritonavir and a second administration at a dosage of 20 mg docetaxel with 200 mg ritonavir. In another embodiment, a bidaily weekly schedule is provided for the treatment of cancer, wherein docetaxel is administered on one day a week a first administration at a dosage of 20 mg docetaxel with 200 mg ritonavir and a second administration at a dosage of 20 mg docetaxel with 200 mg ritonavir. In still another embodiment, a bidaily weekly schedule is provided for the treatment of cancer, wherein docetaxel is administered on one day a week a first administration at a dosage of 20 mg docetaxel with 100 mg ritonavir and a second administration at a dosage of 20 mg docetaxel with 100 mg ritonavir. In another embodiment, a bidaily weekly schedule is provided for the treatment of cancer, wherein docetaxel is administered on one day a week a first administration at a dosage of 20 mg docetaxel with 200 mg ritonavir and a second administration at a dosage of 20 mg docetaxel with 100 mg ritonavir.

In a further embodiment, docetaxel is administered orally in the treatment of mCRPC at weekly dosage of 50 mg, as such a dosage as shown in the example section can provide for docetaxel exposure levels in tumor tissues that are comparable or at least comparable to a standard of care treatment for docetaxel. Such an administration is preferably at a bidaily weekly schedule. As shown in the example section, the CYP3A inhibitor dosage needed to be adapted for mCRPC patients such that the defined AUC could be obtained. Hence, a bidaily weekly schedule for the treatment of mCRPC is provided herein for the treatment of cancer, wherein docetaxel is orally administered on the same day a first administration at a dosage of 30 mg docetaxel with 200 mg ritonavir and a second administration at a dosage of 20 mg docetaxel with 100 mg ritonavir.

As outlined above, the current invention provides for methods and uses of combinations of docetaxel and a CYP3A inhibitor, for the treatment of cancer. Such methods and uses allow to provide for obtaining docetaxel exposure levels of the tumor tissue comparable to a standard of care treatment for docetaxel, by providing a suitable dosage of docetaxel and/or suitable dosage of the CYP3A inhibitor. As also outlined above, the current invention provides for methods and uses of combinations of docetaxel and a CYP3A inhibitor, for the treatment of cancer. Such methods and uses allow to provide for obtaining docetaxel exposure levels of the tumor tissue at least comparable to a standard of care treatment for docetaxel, by providing a suitable dosage of docetaxel and/or suitable dosage of the CYP3A inhibitor.

The current invention also provides for means and methods to determine suitable dosages of docetaxel and/or the CYP3A inhibitor, and/or monitoring that suitable dosages are used throughout the treatment.

As outlined herein, the docetaxel levels in subjects may be controlled before treatment and/or monitored and controlled in treatment following the use of oral administration of docetaxel and CYP3A inhibitor. Such monitoring and control may alternatively (or additionally) also be exerted by measuring docetaxel in blood plasma. Such monitoring and control may also be exerted by monitoring side effects. As described above, docetaxel clearance in a subject may vary due to unknown causes in addition to CYP3A activity. Hence, monitoring docetaxel levels in subjects during treatment allows one to adapt docetaxel dosages to maintain appropriate levels of docetaxel in the subjects. Monitoring side effects assists therein as well. As shown in the example section, when an area under the curve as determined in a standard of care treatment is provided, one can determine the suitable dosages of a CYP3A inhibitor and docetaxel combination that can achieve exposure levels of tumor tissue which are comparable to the standard of care treatment. As shown in the example section, a selected docetaxel and CYP3A inhibitor combination treatment, monitoring the plasma levels in mCRPC patients, indicated that in order to achieve the defined area under the curve, the treatment needed to be adapted. In the first adaptation, the area under the curve increase was too high, resulting in undesired side effects, upon the second adaptation, the defined area under the curve was achieved, providing for the defined exposure levels of tumor tissue while having significantly reduced side effects.

Accordingly, the current invention also provides for a method for the treatment of a cancer, comprising a combination of CYP3A inhibitor and orally administered docetaxel, comprising the steps of:
administering a combination of the CYP3A inhibitor and docetaxel;
determining plasma levels of docetaxel in the subject;
optionally, comparing the levels of docetaxel to a reference level;
determining the docetaxel dosage for administration of a subsequent combination of the CYP3A inhibitor and docetaxel; and
administering the subsequent combination of the CYP3A inhibitor and docetaxel.

By determining the plasma levels of docetaxel after the first administration, it can be confirmed that the selected dosages of the first administration (of both the CYP3A inhibitor and docetaxel) are suitable dosages. Conversely, when plasma levels are too high or low, the dosages of the subsequent combination of the CYP3A inhibitor and docetaxel may be adjusted. Either the CYP3A inhibitor or docetaxel dosage or both CYP3A inhibitor and docetaxel dosage may be adjusted. Hence, the CYP3A inhibitor dosage may remain the same at the first administration and subsequent dosages, and docetaxel dosage adjusted to compensate for any increase or decrease in docetaxel plasma level as compared with a standard of care treatment for docetaxel. Accordingly, in such a method of treatment in accordance with the invention involving determining docetaxel plasma levels, the CYP3A inhibitor is administered at a pre-determined dosage. Also, the docetaxel dosage may remain the same at the first administration and subsequent dosages and the and CYP3A dosage adjusted to compensate for any increase or decrease in docetaxel plasma level as compared with a standard of care treatment for docetaxel. Preferably, the dosage of docetaxel is to be adjusted in order to eradicate the tumor cells. This way, throughout the treatment, the dosage of docetaxel of at least the subsequent combination of the CYP3A inhibitor and docetaxel is sufficient to obtain docetaxel exposure levels of the tumor tissue comparable or at least comparable to a standard of care treatment for docetaxel. Preferably, the said method for the treatment of a cancer comprises multiple administrations of a combination of the CYP3A inhibitor and docetaxel, wherein after each administration the levels of docetaxel are determined, for determining the docetaxel dosage for administration of a subsequent combination of the CYP3A inhibitor and docetaxel. Hence, when the level of docetaxel is increased in the subject as compared with the reference level, the dosage of docetaxel is reduced, and wherein when the level of docetaxel is decreased during treatment as compared with a reference level, the dosage of docetaxel is increased, as compared with the previous dosage administered.

In another embodiment, the invention provides for a method for the treatment of a cancer, comprising a combination of CYP3A inhibitor and orally administered docetaxel, comprising the steps of:
determining the activity of CYP3A in a subject;
optionally, comparing the activity of CYP3A to a reference level;
determining the CYP3A inhibitor dosage based on the activity level of CYP3A determined in the subject;
administering a combination of the CYP3A inhibitor at the determined dosage and docetaxel,
wherein the dosage of docetaxel is sufficient to obtain docetaxel exposure levels of the tumor tissue comparable to a standard of care treatment for docetaxel.

As the activity of CYP3A in a subject may have an effect on the dosage of docetaxel that is to be administered to obtain sufficient exposure levels, determining the activity of CYP3A may have the advantage that possible variation between subjects can be taken into account. This can be done before the treatment commences. The CYP3A activity in a subject can be determined by any known means, but also by measuring plasma levels of ritonavir or other indirect methods. By knowing before treatment commences what an appropriate dose of the CYP3A inhibitor is, from the initiation of treatment, immediately the desired exposure levels of docetaxel in the subject can be obtained. Hence, in a preferred embodiment, in a method in accordance with the invention the steps of determining the activity, subsequent optional comparison step, and determining dosage step for the CYP3A inhibitor, are carried out prior to the first administration of the combination of the CYP3A inhibitor and docetaxel. More preferably, docetaxel is administered at a pre-determined dosage. When for example the CYP3A activity is relatively high before treatment, the dosage of the CYP3A inhibitor can be selected to be relatively higher, and when the CYP3A activity is low, the dosage can be selected to be relatively lower. It is understood that the CYP3A activity may also change during the treatment with the combination of oral docetaxel and the CYP3A inhibitor. For example, when the cancer that is the subject of the therapy comprises substantial CYP3A activity (Hendrikx et al., Int J Cancer, 2015; Ikezoe et al., Cancer Res, 2004), in treatment, because of the reduction of the cancer, the CYP3A activity in a subject may reduce as well, requiring less of a CYP3A inhibitor to maintain an efficacious level of docetaxel. Accordingly, in this method of treatment, the steps of the method can be carried out during the treatment comprising the administration of the combination of the CYP3A inhibitor and docetaxel. Hence, in a further embodiment, in the method of treatment involving determining the CYP3A activity in a subject, when the activity of CYP3A is increased during treatment, the dosage of the CYP3A inhibitor is increased, and wherein when the activity of CYP3A is decreased during treatment, the dosage of the CYP3A inhibitor is maintained or reduced as compared with the previous dosage administered. This way, optimal exposure levels in subjects can be obtained avoiding too high or too low levels of docetaxel after oral administration of docetaxel and a CYP3A inhibitor.

As is clear from the above, docetaxel plasma levels can be controlled by determining or monitoring CYP3A activity in a subject and/or by monitoring docetaxel plasma levels in a subject, and by subsequently using that information to adjust CYP3A inhibitor dosage and/or docetaxel for oral administration dosage, if necessary, to obtain docetaxel exposure levels of the tumor tissue which are comparable to a standard of care treatment for docetaxel. Hence the methods as described above which involve measuring CYP3A activity or docetaxel plasma levels in subjects, are not restricted to measuring only CYP3A activity or docetaxel, but may also comprise measuring both docetaxel and CYP3A activity. Hence, when e.g., docetaxel levels in a subject are varying due to (unknown) causes other than CYP3A activity, better control can be exerted on docetaxel exposure levels in subjects because in such situations it may be preferred to adjust docetaxel dosages instead of varying the dosage of CYP3A inhibitor. Hence, a further method is provided wherein in a method for the treatment of a cancer is provided, said method comprising a combination of CYP3A inhibitor and orally administered docetaxel, which comprises the steps of:
determining the activity of CYP3A in a subject;
optionally, comparing the activity of CYP3A to a reference level;
optionally, determining the CYP3A inhibitor dosage based on the activity level of CYP3A determined in the subject;
administering a combination of the CYP3A inhibitor and docetaxel;
determining plasma levels of docetaxel in the subject;
optionally, comparing the levels of docetaxel to a reference level;
determining the docetaxel dosage and/or CYP3A inhibitor dosage for administration of a combination of the CYP3A inhibitor and docetaxel based on the CYP3A activity and docetaxel levels determined.

It is understood that as described above, the activity of CYP3A and docetaxel plasma levels may be carried out in treatment only, i.e., after the first administration of the combination of CYP3A inhibitor and docetaxel. It is also understood that the method of treatment considers first CYP3A activity of a subject, resulting in selecting first dosages of docetaxel and CYP3A inhibitor, followed by subsequent in treatment monitoring of CYP3A activity and/or docetaxel plasma levels to determine suitable dosages for CYP3A inhibitor and/or orally formulated docetaxel.

In further embodiments, kits are provided that are for use in the methods and uses as described herein for the combinations of docetaxel and the CYP3A inhibitor. In one embodiment, a kit is provided comprising a pharmaceutical composition comprising docetaxel for oral administration and a pharmaceutical composition comprising a CYP3A inhibitor. In another embodiment, a kit is provided
comprising a pharmaceutical composition comprising docetaxel for oral administration and a pharmaceutical composition comprising a CYP3A inhibitor, wherein said kit is for the treatment of a solid tumor, in particular non-small cell-lung cancer, gastric cancer, a breast cancer, a head and neck cancer or a prostate cancer, more in particular mCRPC. In still another embodiment, a kit is provided
comprising a pharmaceutical composition comprising docetaxel and a pharmaceutical composition comprising a CYP3A inhibitor, wherein said kit is for use in a combination therapy as defined in any one of the methods and uses in accordance with the invention as described herein.

Pharmaceutical compositions of this invention may comprise docetaxel, or pharmaceutically acceptable salts and esters thereof, and/or a CYP3A inhibitor, such as ritonavir, (or pharmaceutically acceptable salts and esters thereof) together with any pharmaceutically acceptable carrier, adjuvant or vehicle.

As used in the description of the invention, clauses and clauses appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

### Examples

### Modradoc006

Modradoc006 is a spray-dried solid dispersion formulation of docetaxel pressed into tablets (ModraDoc006 10 mg tablets), containing 10 mg docetaxel. The formulation excipients are polyvinyl pyrrolidone K30, sodium dodecyl sulphate, lactose monohydrate, croscarmellose, silica colloidalis anhydrica and magnesium stearate. All excipients are included in the FDA guide for inactive compounds (oral capsules and tablets).

### Ritonavir

Ritonavir is commercially available as 100 mg tablets for oral consumption (Norvir®). This tablet has been granted approval by the European Commission in 2010.

### Docetaxel and Ritonavir Plasma Measurements

A combined assay for the determination of docetaxel and ritonavir in human plasma is described. The drugs were extracted from 200 μL human plasma using liquid-liquid extraction with tertiary-butylmethylether, followed by high performance liquid chromatography analysis using 10 mM ammonium hydroxide pH 10:methanol (3:7, v/v) as mobile phase. Chromatographic separation was obtained using a Zorbax Extend C(18) column. Labelled analogues of the analytes are used as internal standards. For detection, positive ionization electrospray tandem mass spectrometry was used. Method development including optimisation of the mass transitions and response, mobile phase optimisation and column selection are discussed. The method was validated according to FDA guidelines and the principles of Good Laboratory Practice (GLP). The validated range was 0.5-500 ng/mL for docetaxel and 2-2000 ng/mL for ritonavir. For quantification, quadratic calibration curves were used (r(2)>0.99). The total runtime of the method is 9 min and the assay combines analytes with differences in ionisation and desired concentration range. Inter-assay accuracy and precision were tested at four concentration levels and were within 10% and less than 10%, respectively, for all analytes. Carry-over was less than 6% and endogenous interferences or interferences between analytes and internal standards were less than 20% of the response at the lower limit of quantification level. The matrix factor and recovery were determined at low, mid and high concentration levels. The matrix factor was around 1 for all analytes and total recovery between 77.5 and 104%. Stability was investigated in stock solutions, human plasma, dry extracts, final extracts and during 3 freeze/thaw cycles. The described method was successfully applied in clinical studies with oral administration of docetaxel in combination with ritonavir.

mCRPC Trial

In a phase I trial we investigated oral treatment with ModraDoc006/r in patients with several solid tumors (not prostate). From this study it was concluded that the recommended dose for phase II efficacy evaluation is:

ModraDoc006 30 mg+Ritonavir 100 mg, taken simultaneously in the morning

ModraDoc006 20 mg+Ritonavir 100 mg, taken simultaneously in the evening

This treatment (denoted as ModraDoc006/r 30-20/100-100) is given on one day, once every week.

Pharmacokinetics research revealed that the docetaxel AUC0-48 h of this treatment schedule in cycle 1 is: 1126±382 h*ng/mL. The CMAX value was 102±46 ng/mL (average of 16 treated patients).

Our next step was to investigate this oral treatment schedule in patients with metastatic Castration Resistant Prostate Cancer (mCRPC) in a phase IB/IIA trial (M17DOC). Surprisingly, in the first 5 patients treated with the recommended dose from the phase I trial (ModraDoc006/r 30-20/100-100), we noted a much lower docetaxel exposure (AUC0-48 h) of 498±298 h·ng/mL, approximately half than expected. The CMAX values: 45±31 ng/mL, were also half than expected. Patients experienced no notable side effects. It was concluded that docetaxel has a higher clearance in this mCRPC patient population than in patients with other solid tumors.

We then hypothesized that we could achieve our targeted exposure of around 1100±500 h·ng/mL by increasing (doubling) the CYP3A inhibitor ritonavir dose. Eight mCRPC patients were then treated with ModraDoc006/r 30-20/200-200 (taken on one day, once per week). The docetaxel exposure in this cohort was: AUC0-48 h: 2032±1018 h·ng/mL and CMAX 164±80 ng/mL. These values were higher than expected. Patients also experienced more side effects (grade III).

Next we treated mCRPC patients (n=3) with the dose ModraDoc006/r 30-20/200-100 in the assumption that with a reduced ritonavir dose the docetaxel exposure would decrease towards its targeted value. In this treated cohort of patients the docetaxel exposure was AUC0-48 h: 1130±257 h·ng/mL and CMAX 135±46 ng/mL. Treatment is well tolerated.

Results are also depicted in FIGS. 2A and 2B . . . .

To summarize:

| | docetaxel | docetaxel |
|---|---|---|
| | AUC0-48 h | CMAX |
| ModraDoc006/r | (h · ng/mL)¶ | (ng/mL)¶ |
| 30-20/100-100 (phase I →target) | 1100 | 100 |
| 30-20/100-100 (phase IB/IIA mCRPC) | 500 | 45 |
| 30-20/200-200 (phase IB/IIA mCRPC) | 2000 | 165 |
| 30-20/200-100 (phase IB/IIA mCRPC) | 1100 | 135 |

¶Median values (for cycle 1) are rounded off and with variation that may occur up to 40-50%.

The trial results described above were obtained during the trial and represent intermediate results. The trial continued and below updated results are described.

Phase IB/IIA Study in mCRPC

A multicenter clinical phase IB/IIA study was conducted in mCRPC (M17DOC), wherein ModraDoc006, an oral docetaxel formulation was combined with ritonavir (ModraDoc006/r), in metastasized castration-resistant prostate cancer (mCRPC).

The study included patients diagnosed with metastasized castration-resistant prostate cancer (mCRPC), dosed in a bi-daily once weekly (BIDW) dosing schedule at 4 dose levels (see table below).

| Dose level | ModraDoc006 | Ritonavir | # patients |
|---|---|---|---|
| −2A | 30 mg/20 mg | 100 mg/100 mg | 5 |
| 1A | 30 mg/20 mg | 200 mg/200 mg | 6 |
| −1A | 30 mg/20 mg | 200 mg/100 mg | 6 |
| −2B | 20 mg/20 mg | 200 mg/100 mg | 3 |

As said, surprisingly, in the first 5 patients treated with the recommended dose from the phase I trial (ModraDoc006/r 30-20/100-100), a much lower docetaxel exposure in cycle 1 (median AUC0-48 h±SD) of 454±181 h·ng/mL, approximately half than expected. The CMAX values: 38±18 ng/mL, were also half than expected. Patients experienced no notable side effects. It was concluded that docetaxel has a higher clearance in this mCRPC patient population than in patients with other solid tumors.

As said, we then hypothesized that we could achieve our targeted exposure of around 1100±500 h·ng/mL by increasing (doubling) the CYP3A inhibitor ritonavir dose. Eight patients mCRPC, of which six were evaluable, were then treated with ModraDoc006/r 30-20/200-200 (taken on one day, once per week). The docetaxel exposure in this cohort in cycle 1 was: median AUC0-48 h±SD 1510±990 h·ng/mL and CMAX 146±82 ng/mL. These values were higher than expected. Patients also experienced more side effects (grade III).

Next we treated mCRPC patients (n=6) with the dose ModraDoc006/r 30-20/200-100 in the assumption that with a reduced ritonavir dose the docetaxel exposure would decrease towards its targeted value. In this treated cohort of patients the docetaxel exposure was in cycle 1: median AUC from 0-48 h±SD 1189±473 h·ng/mL and CMAX 159±49 ng/mL. Treatment is well tolerated.

In mCRPC patients (n=3) treated with ModraDoc006/r 20-20/200-100, with thus a reduced docetaxel morning dose, the docetaxel exposure was in cycle 1: median AUC from 0-48 h±SD 419±158 h·ng/mL and CMAX 53±21 ng/mL.

To summarize:

| ModraDoc006/r | docetaxel AUCO-48 h(h.ng/mL)¶ | docetaxel CMAX (ng/mL)¶ |
|---|---|---|
| 30-20 / 100-100 (prior phase I →target) | 1100 | 100 |
| 30-20 / 100-100 (phase IB/IIA mCRPC) | 500 | 40 |
| 30-20 / 200-200 (phase IB/IIA mCRPC) | 1500 | 150 |
| 30-20 / 200-100 (phase IB/IIA mCRPC) | 1200 | 160 |
| 20-20 / 200-100 (phase IB/IIA mCRPC) | 420 | 50 |

¶Median values for cycle 1 are rounded off and with variation that may occur up to 40-50%.

Results of the trial are further listed below and depicted in FIGS. 4 to 8.

| | | | | |
|---|---|---|---|---|
| | total # | PSA | Best PSA | Radiological |
| Patient # | cycles | Change % | response | response |

Dose level −2A

| | | | | |
|---|---|---|---|---|
| #0101 | 8 | −11% | PSA equal to baseline or decline | non CR/non PD |
| | | | <50% | |
| #0102 | 7 | 19% | PSA increase | SD |
| #0103 | 6 | 144% | PSA increase | non CR/non PD |
| #0104 | 5 | −6% | PSA equal to baseline or decline | — |
| | | | <50% | |
| #0105 | 2 | −11% | PSA equal to baseline or decline | — |
| | | | <50% | |

Dose level 1A

| | | | | |
|---|---|---|---|---|
| #0301 (NE) | 1 | −17% | PSA equal to baseline or decline | NE |
| | | | <50% | |
| #0106 | 8 | 44% | PSA increase, suspected | non CR/non PD |
| | | | progression | |
| #0302 | 12 | −53% | PSA response, non-confirmed | SD |
| #0201 (NE) | 2 | −10% | PSA equal to baseline or decline | NE |
| | | | <50% | |
| #0401(DLT) | 4 | −40% | PSA equal to baseline or decline | — |
| | | | <50% | |
| #0303 | 5 | 84% | PSA increase, suspected flare-up | SD (after 5 |
| | | | | cycles) |
| #0402 | 30 | −98% | PSA response, confirmed | non CR/non PD |
| #0202 (DLT) | 3 | −70% | PSA response, confirmed | SD (after 3 |
| | | | | cycles) |

Dose level −1A

| | | | | |
|---|---|---|---|---|
| #0304 | 11 | 14% | PSA equal to baseline or decline PD | |
| | | | <50% | |
| #0403 | 17 | −33% | PSA equal to baseline or decline non CR/non PD | |
| | | | <50% | |
| #0404 | 30 | 61% | PSA increase, suspected flare-up non CR/non PD | |
| #0109 (NE) | 2 | −31% | PSA equal to baseline or decline NE | |
| | | | <50% | |
| #0306 | 12 | −60% | PSA response, non-confirmed | SD |
| #0307(DLT) | 3 | 0% | PSA equal to baseline or decline | — |
| | | | <50% | |
| #0406 | 27 | −95% | PSA response, confirmed | non CR/non PD |

Dose level −2B

| | | | | |
|---|---|---|---|---|
| #0107 | 30 | −97% | PSA response, confirmed | PR |
| #0305 (NE) | 0 | | | NE |
| #0108 | 30 | −34% | PSA equal to baseline or decline | non CR/non PD |
| | | | <50% | |
| #0405 | 30 | −91% | PSA response, confirmed | PR |

(PSA (prostate specific antigen);; SD (Stable disease); non CR(non complete response); non PD(non-progressive disease); PD (progressive disease); NE (non evaluable); PR (partial response)).

### Efficacy Summary:

This study included 20 evaluable patients diagnosed with metastasized castration-resistant prostate cancer (mCRPC), dosed in a bi-daily once weekly (BIDW) dosing schedule at 4 dose levels (see table). In 7 patients PSA response (PSA decline ≥50%) was observed, of which 5 were confirmed by a second measurement after 6 weeks. In another 7 patients PSA declined <50% or remained equal to baseline. In the remaining 6 patients PSA increase was observed. Despite PSA decline <50% in one patient and PSA increase in another patient, noticeable clinical response with pain reduction was achieved during the maximum treatment duration of 30 weeks. A total of 5 patients completed the maximum of 30 treatment weeks. The median treatment duration was 14 weeks. ModraDoc006/r 30-20/200-100 is a preferred initial dose to be further tested in mCRPC, given it demonstrated the ability to achieve exposure levels of docetaxel (as measured by AUC) which were higher than achieved with IV docetaxel, while also having acceptable toxicity. Alternatively, ModraDoc006/r 20-20/200-100 may be another preferred dose, or preferred initial dose, in mCRPC.

### Extended Use

### N07DOW

We treated in a phase I trial (N07DOW) cancer patients (n=100) with oral docetaxel in combination with ritonavir. The dose was administered on one day (single dose), once per week. Data presented as mean±standard deviation. If available, kinetic data of 2 cycles per patient were used.

Treatment duration of 19 patients was from 19 up to 72 weeks. These were patients with the following cancers. Head and neck (n=1), non-small cell lung (n=8), anal (n=1), primary unknown (n=3), ovarian (n=1), esophageal (n=1), urothelial cell (n=2), leiomyosarcoma (n=1) and neuroendocrine lung carcinoma (n=1). The docetaxel exposure in these patients was:

| | |
|---|---|
| AUCO-48 h | 803 ± 634 h.ng/mL |
| CMAX (peak) | 148 ± 113 ng/mL |

SAEs (Serious Adverse Events) and DLTs (Dose-Limiting Toxicity) (possible, probable, definite; ≥grade 3) were noted in 15 patients. The docetaxel exposure in these patients was:

| | |
|---|---|
| AUCO-48 h | 2345 ± 1453 h.ng/mL |
| CMAX | 351 ± 244 ng/mL |

Fiftytwo patients had SD (stable disease) (n=42) or PR (partial response) (n=10) as best treatment response. The docetaxel exposure in these patients was:

| | |
|---|---|
| AUCO-48 h | 1083 ± 1023 h.ng/mL |
| CMAX | 197 ± 186 ng/mL |

N10BOM

We treated in a phase I trial (N10BOM) cancer patients (n=64) with oral docetaxel in combination with ritonavir. The dose was administered bi-daily once per week continuously.

Treatment duration of 8 patients was from 19 up to 55 weeks. These were patients with the following cancers. Head and neck cancer (n=2; PR), non-small cell lung (n=4; SD), colorectal (n=1;SD) and giant cell neuroendocrine carcinoma (n=1;SD). The docetaxel exposure in these patients was:

| | | |
|---|---|---|
| AUCO-48 h | 1 | 224 ± 620 h.ng/mL |
| CMAX | | 143 ± 67 ng/mL |

SAEs and DLTs (possible, probable, definite; ≥grade 3) were noted in 10 patients. The docetaxel exposure in these patients was:

| | |
|---|---|
| AUCO-48 h | 1809 ± 1255 h.ng/mL |
| CMAX | 175 ± 117 ng/mL |

Twenty-five patients had SD or PR as best treatment response. The docetaxel exposure in these patients was:

| | |
|---|---|
| AUCO-48 h | 1242 ± 702 h.ng/mL |
| CMAX | 140 ± 83 ng/mL |

To summarize

Treatment duration 19 weeks and longer:

| | N07DOW | N10BOM |
|---|---|---|
| AUCO-48 h | 803 ± 634 h.ng/mL | 1224 ± 620 h.ng/mL |
| CMAX | 148 ± 113 ng/mL | 143 ± 67 ng/mL |

SAEs and DLTs

| | N07DOW | N10BOM |
|---|---|---|
| AUCO-48 h | 2345 ± 1453 h.ng/mL | 1809 ± 1255 h.ng/mL |
| CMAX | 351 ± 244 ng/mL | 175 ± 117 ng/mL |

SDs and PRs Responses

| | N07DOW | N10BOM |
|---|---|---|
| AUCO-48 h | 1083 ± 1023 h.ng/mL | 1242 ± 702 h.ng/mL |
| CMAX | 197 ± 186 ng/mL | 140 ± 83 ng/mL |

For comparison:

A weekly administration of docetaxel (35 mg/m2) as a 0.5 h intravenous infusion gives the following AUC and CMAX-values.

| | |
|---|---|
| AUC | 480 ± 410 h.ng/mL |
| CMAX | 1930 ± 600 ng/mL |

Baker S D et al. Clin Cancer Res 2004; 10:1976-1983.

For bi-daily, once per week use of oral docetaxel with ritonavir (ModraDoc006/r) the following targeted values may be proposed:

| | |
|---|---|
| AUC | 1200 ± 600 h.ng/mL |
| CMAX | 140 ± 70 ng/mL |

With this weekly oral treatment schedule similar docetaxel exposure (AUC) is achieved on the administration day, as on the administration day of the weekly intravenous treatment schedule (moreover, intravenously often given as 3 consecutive weeks followed with 1 week rest while oral docetaxel is given continuously with no rest week). CMAX values after this intravenous administration (35 mg/m2 in 0.5 h) are ten-fold higher than after oral ModraDoc006/r 30-20/100-100 in patients with solid tumors (not prostate).
Intravenous (35 mg/m2) docetaxel and oral docetaxel treatment (ModraDoc006/r 30-20/100-100) give similar AUCs and thus comparable efficacy is to be expected;
Intravenous (35 mg/m2 in 0.5 h) docetaxel gives a ten-fold higher CMAX than oral docetaxel treatment (ModraDoc006/r 30-20/100-100) which may explain higher toxicity for the intravenous treatment;
In oral docetaxel treatment (ModraDoc006/r) higher AUC0-48 h-CMAX values correlate with toxicity;
For oral docetaxel treatment (ModraDoc006/r) an AUC0-48 h of 1200±600 h·ng/mL seems optimal and which can be achieved in cancer patients with solid tumors (not prostate) by a bi-daily weekly schedule with ModraDoc006/r 30-20/100-100.

### Phase IIA Study in Breast Cancer

A multicenter clinical phase IIA study was conducted in metastatic breast cancer (M18DMB), wherein ModraDoc006, an oral docetaxel formulation was combined with ritonavir (ModraDoc006/r), in patients with recurrent or metastatic HER-2 negative breast cancer suitable for treatment with a taxane. Results of the trial are summarized below and depicted in FIGS. 9 and 10.

| | Tumor | Total | Best | |
|---|---|---|---|---|
| | measurements | #of | response | Overall |
| Patient # | (min) | cycles | (%) | Response |
| 001 | 58-48.4 | 8 | −17% | PD |
| 002 | 16 | 2 | | NE |
| 003 | 22.6-15.2-12.3 | 12 | −46% | PR-c |
| 004 | 49-51-53-59 | 18 | 4% | SD |
| 005 | 47-48-55 | 12 | 2% | SD |
| 006 | 68-60-51 | 12 | −25% | SD |
| 007 | — | | | |
| 008 | 22-22 | 6 | 0% | SD |
| 009 | 37-28 | 6 | −24% | SD |
| 010 | 79-64-35 | 22 ong | −66% | PR-c |
| 011 | 68-47 | 20 ong | −38% | PR-c |
| 012 | 29 | 7 | | NE |
| 013 | 77-73-63 | 12 | −18% | SD |

(PD (progressive disease); NE (Non-evaluable); PR-c (confirmed Partial Response), ong (ongoing).

Tumor measurements represent changes in tumor size over time as measured by CT scan, with the initial value being baseline.

### Efficacy Summary:

A total of 12 patients with recurrent or metastatic breast cancer, suitable for treatment with a taxane, were treated in this study at a bi-daily once weekly (BIDW) dosing schedule with thirty (30) mg ModraDoc006 combined with 100 mg ritonavir (/r) in the morning and 20 mg ModraDoc006 with 100 mg/r in evening. In 10 patients evaluable for efficacy (i.e., they received a minimum of 6 weekly treatments and with disease assessments according to RECIST 1.1), responses resulted in 3 confirmed (repeated tumor measurement after >4 weeks) partial responses (PR), 6 stable disease (SD) and 1 progressive disease (PD). Median treatment duration in 12 patients is currently 11.3 weeks, with 2 patients still ongoing at respectively 20 and 22 weeks.

1. A method for the treatment of cancer, the method comprising orally administering docetaxel in combination with a CYP3A inhibitor.

2. The method of claim 1, wherein
the dose of docetaxel is adjusted to compensate for increased clearance of docetaxel in subjects having cancer;
the dose of the CYP3A inhibitor is sufficient to substantially diminish the increased clearance of docetaxel in subjects having mCRPC; and/or
the administration of the CYP3A inhibitor in combination with the docetaxel is sufficient to obtain docetaxel exposure levels in the tumor tissue which are comparable to a standard of care treatment for docetaxel.

3. The method of claim 1, wherein said cancer is a solid tumor.

4. The method of claim 3, wherein said tumor is a non small cell lung cancer, a gastric cancer, a breast cancer, a head and neck cancer or a prostate cancer.

5. The method of claim 4, wherein said prostate cancer is metastatic castration-resistant prostate cancer (mCRPC).

6.-10. (canceled)

11. The method of claim 1, wherein said CYP3A inhibitor is ritonavir.

12. The method of claim 1, wherein the method does not comprise the use of prednisone.

13. The method of claim 1, wherein the docetaxel is administered orally in a weekly dosage of 50 mg.

14. The method of claim 1, wherein the CYP3A inhibitor comprises ritonavir and is administered in a weekly dosage in the range of 200-300 mg.

15. The method of claim 1, wherein the dose of the CYP3A inhibitor is sufficient to obtain docetaxel exposure levels of the tumor tissue which is comparable to a standard of care treatment of docetaxel given intravenously every three weeks.

16.-17. (canceled)

18. A method for the treatment of a cancer, comprising the steps of:
determining the activity of CYP3A in a subject;
optionally, comparing the activity of CYP3A to a reference level;
optionally, determining the CYP3A inhibitor dosage based on the activity level of CYP3A determined in the subject;
administering a combination of the CYP3A inhibitor, optionally at the determined dosage and the docetaxel,
wherein the dosage of docetaxel is optionally sufficient to obtain docetaxel exposure levels of the tumor tissue comparable to a standard of care treatment for docetaxel.

19. (canceled)

20. The method according to claim 18, wherein the determining steps of the method are carried out prior to the first administration of the combination of the CYP3A inhibitor and docetaxel.

21. The method according to claim 18, wherein the steps of the method are carried out during the treatment comprising administration of the combination of the CYP3A inhibitor and docetaxel.

22. The method according to claim 21, wherein when the activity of CYP3A is increased during treatment, the dosage of the CYP3A inhibitor is increased, and wherein when the activity of CYP3A is decreased during treatment, the dosage of the CYP3A inhibitor is maintained or reduced as compared with the previous dosage administered.

23. A method for the treatment of a cancer, comprising a combination of CYP3A inhibitor and orally administered docetaxel, comprising the steps of:
administering a combination of the CYP3A inhibitor and docetaxel;
determining plasma levels of docetaxel in the subject;
optionally, comparing the levels of docetaxel to a reference level;
determining the docetaxel dosage for administration of a subsequent combination of the CYP3A inhibitor and docetaxel; and
administering the subsequent combination of the CYP3A inhibitor and docetaxel.

24. The method according to claim 23, wherein the dosage of docetaxel of at least the subsequent combination of the CYP3A inhibitor and docetaxel is sufficient to obtain docetaxel exposure levels of the tumor tissue comparable to a standard of care treatment for docetaxel.

25. The method according to claim 23, wherein the method for the treatment of a cancer comprises multiple administrations of a combination of the CYP3A inhibitor and docetaxel, wherein after each administration the levels of docetaxel are determined, for determining the docetaxel dosage for administration of a subsequent combination of the CYP3A inhibitor and docetaxel.

26.-27. (canceled)

28. The method of claim 18 further comprising the steps of:
determining plasma levels of docetaxel in the subject;
optionally, comparing the levels of docetaxel to a reference level; and/or
determining the docetaxel dosage and/or CYP3A inhibitor dosage for administration of a combination of the CYP3A inhibitor and docetaxel based on the CYP3A activity and docetaxel levels determined.

29.-30. (canceled)

31. A kit for use in the method of claim 1, the kit comprising:
a pharmaceutical composition comprising Docetaxel; and
a pharmaceutical composition comprising a CYP3A inhibitor.
